(19) United States
(12) Patent Application Publication
(10) Pub. No.: US 2007/0299031 A1
(43) Pub. Date: Dec. 27, 2007

(54) THERAPEUTIC AGENT FOR TUMOR OF NEURAL ORIGIN

(75) Inventors: Hideyuki OKANO, Tokyo (JP); Wado Akamatsu, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: JAPAN SCIENCE AND TECHNOLOGY AGENCY, Kawaguchi-shi (JP)

(21) Appl. No.: 11/771,083

(22) Filed: Jun. 29, 2007

Related U.S. Application Data
(62) Division of application No. 10/478,514, filed on Nov. 24, 2003.

(30) Foreign Application Priority Data
May 24, 2001 (JP) 2001-155237

(51) Int. Cl. A61K 31/711; A61P 35/00

(52) U.S. Cl. 514/44

(57) ABSTRACT

The present invention is directed to a therapeutic agent for a tumor of neural origin, containing, as an active ingredient, any of the following: Hu protein; a polypeptide having an amino acid sequence derived from an amino acid sequence of Hu protein by substitution, deletion, addition, or insertion of one or more amino acid residues; or a gene encoding the amino acid sequence of Hu protein or the peptide. Thus, the present invention provides a novel method for treating neuroblastoma.

### TECHNICAL FIELD

The present invention relates to a therapeutic agent for a tumor of neural origin such as neuroblastoma.

### BACKGROUND ART

Neuroblastoma is a malignant tumor of the ganglion neuronal lineage. It typically develops in children under three years of age and often results in death. Among all solid tumors occurring in children, neuroblastoma has the highest incidence. Many cases of neuroblastoma retracts naturally, but not a few cases are malignant, involving N-myc gene amplification. Since patients are mostly children, who require special considerations, effective therapeutic drugs for treatment of neuroblastoma have not yet been discovered. Thus, a need continues to exist for development of a novel therapeutic method for neuroblastoma.

### DISCLOSURE OF THE INVENTION

The Hu protein is an RNA binding protein that is specifically expressed in differentiated neurons. The present inventors, having been interested in this protein, incorporated Hu protein genes into SH-SY cells, which are a type of neuroblastoma cell, to thereby cause overexpression of Hu protein in the cells, and found that apoptosis of the SH-SY cells was induced and that multiplication of the SH-SY cells was substantially halted. The present invention has been accomplished on the basis of this finding.

Accordingly, the present invention provides a therapeutic agent for a tumor of neural origin, containing, as an active ingredient, any of the following: Hu protein; a polypeptide having an amino acid sequence derived from an amino acid sequence of Hu protein by substitution, deletion, addition, or insertion of one or more amino acid residues; or a gene encoding the amino acid sequence of Hu protein or the peptide.

The present invention also provides use of any of the following in production of therapeutic agents for a tumor of neural origin: Hu protein; a polypeptide having an amino acid sequence derived from an amino acid sequence of Hu protein by substitution, deletion, addition, or insertion of one or more amino acid residues; or a gene encoding the amino acid sequence of Hu protein or the peptide.

The present invention also provides a method for treating a tumor of neural origin, comprising administering an effective amount of any of the following: Hu protein; a polypeptide having an amino acid sequence derived from an amino acid sequence of Hu protein by substitution, deletion, addition, or insertion of one or more amino acid residues; or a gene encoding the amino acid sequence of Hu protein or the peptide.

### BRIEF DESCRIPTION OF THE DRAWINGS

FIGS. 1a and 1d are photomicrographs of HuB-incorporated SH-SY5Y cells; FIGS. 1b and 1e show the results of immunostaining of the HuB-incorporated SH-SY5Y cells; and FIGS. 1cand 1f show the results of Hoechst staining of the HuB-incorporated SH-SY5Y cells.

FIG. 2 is a graph showing TUNEL positive rate (%) of the HuB-incorporated SH-SY5Y cells and that of control cells [TUNEL positive cells/FLAG (or Myc) positive cells].

FIG. 3 shows the results of immunostaining of SH-SY5Y cells performed 48 hours after incorporation of HuB, with an anti-BrdU antibody or with an anti-FLAG (or anti-Myc) antibody (a: stained with anti-BrdU antibody, b: stained with FLAG antibody).

FIG. 4 is a graph showing the BrdU positive rate of the HuB-incorporated cells and that of GFP (control).

FIG. 5 shows the results of immunostaining of the HuB-incorporated SH-SY5Y cells with Bcl-2 antibody (a: stained with Bcl-2 antibody, b: stained with FLAG antibody).

FIG. 6 shows the results of immunoblotting by use of p27 antibody.

FIG. 7 shows a subcloning strategy of UTR-1, UTR-2, and UTR-3 of the human Bcl-2 gene.

FIG. 8 shows a strategy of point mutation in RRM2.

FIG. 9 shows the binding ability between HuB, and Bcl-2 mRNA 3′UTR-1, -2, or -3.

### BEST MODE FOR CARRYING OUT THE INVENTION

Hu protein, serving as the active ingredient of the drug of the present invention, is a protein which has previously been identified as an antigen recognized by an autoantibody that emerges along with neuropathy accompanied by small cell lung carcinoma. Hu protein is an RNA-binding protein which is expressed specifically in differentiated neurons, and has been known to have a function of regulating expression of the target gene product at the post-transcriptional level through binding to the AU-rich element (ARE) on the 3′UTR side of the target mRNA. However, the effect of Hu protein on neuroblastoma has remained completely unknown.

The Hu protein can be isolated from cells in which it is present. Alternatively, the Hu protein can be prepared through DNA recombinant technology from the gene encoding Hu protein, which has already been obtained by use of cloning technology. Specifically, an expression vector is prepared through use of the gene obtained by cloning technology, and cells are transformed with the expression vector, to thereby produce Hu protein.

The Hu protein may be a native protein as expressed in differentiated neurons. Alternatively, the Hu protein may be a modified protein having an amino acid sequence partially modified from that of the native protein, so long as the thus-modified protein has characteristics similar to those of the native protein. For example, there may be used a polypeptide having an amino acid sequence derived from an amino acid sequence of Hu protein by substitution, deletion, addition, or insertion of one or more amino acid residues. No limitations are imposed on the degree of substitution, deletion, addition, or insertion or on positions at which substitution, deletion, addition, or insertion occurs, so long as the polypeptide having such a modified amino acid sequence exhibits characteristics similar to those of Hu protein. The modified polypeptide typically has 80% or more homology with Hu protein, preferably 90% or more homology. As in the case of Hu protein, modified polypeptides may be prepared through DNA recombination techniques.

In the present invention, there may be used a gene therapy in which a gene encoding Hu protein or the above-described modified polypeptide is administered to a patient and the protein or the modified polypeptide is produced in the patient's body. Since such a gene has already been obtained through cloning, use of such a gene is preferred.

As described in the Examples described below, when Hu protein genes are transferred to neuroblastoma-derived SH-SY cells to thereby cause overexpression of Hu protein, apoptosis of the SH-SY cells is induced. In addition, the SH-SY cells in which Hu protein has been overly expressed also stop multiplying. Accordingly, Hu protein or an Hu protein gene is useful as a therapeutic agent for a tumor of neural origin such as neuroblastoma.

Enhancement in p27 expression is considered to play a role in multiplication inhibition of SH-SY cells caused by Hu protein, and suppression of Bcl-2 expression is considered to play a role in apoptosis induction of SH-SY cells. Hu protein is considered to bind to the AU-rich element in the 3′UTR of Bcl-2 mRNA, whereby stability of mRNA is deteriorated, leading to reduction in expression of Bcl-2. The reduction of expression of Bcl-2, which exhibits apoptosis inhibitory effect, is considered to elevate the sensitivity of the SH-SY cells for apoptosis-inducing stimulus, thereby promoting induction of apoptosis.

When administered to mammals, including humans, the drug of the present invention may be formed into a medical composition containing the active ingredient and a pharmaceutically acceptable carrier, and the resultant medical composition may be administered in various dosage forms. A preferred dosage form is injection. Examples of the pharmaceutically acceptable carrier include distilled water, solubilizers, stabilizers, emulsifiers, and buffers. The dose of the drug of the present invention differs depending on the patient's pathological condition, sex, and body weight, etc. The daily dose of Hu protein or Hu protein gene may be about 0.1 μg to about 10 mg.

### EXAMPLES

The present invention will next be described in detail by way of examples, which should not be construed as limiting the invention thereto.

### Example 1

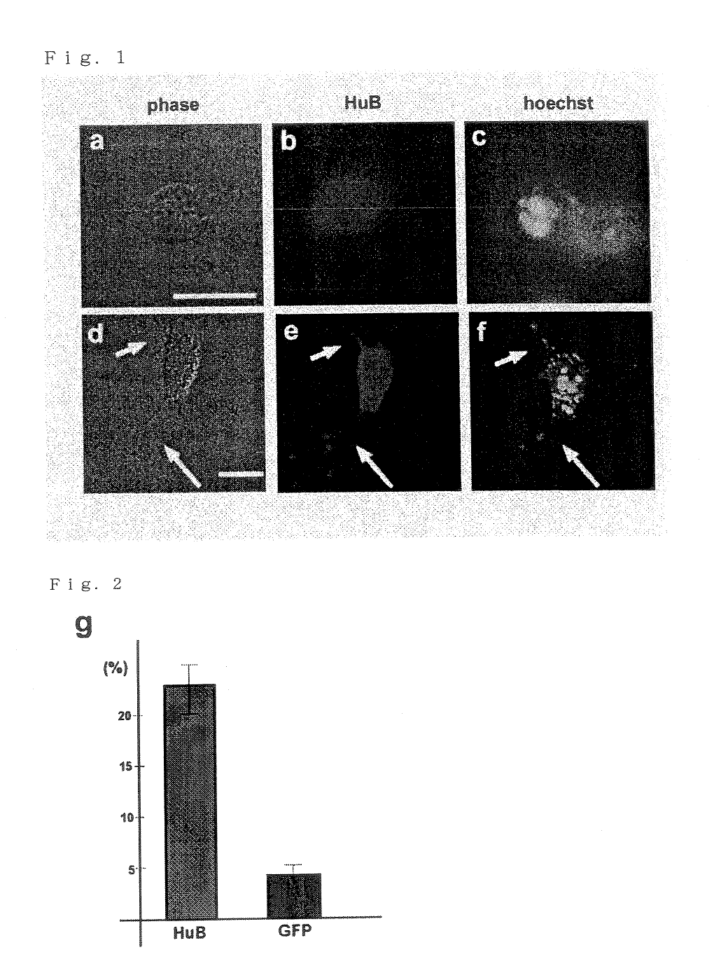

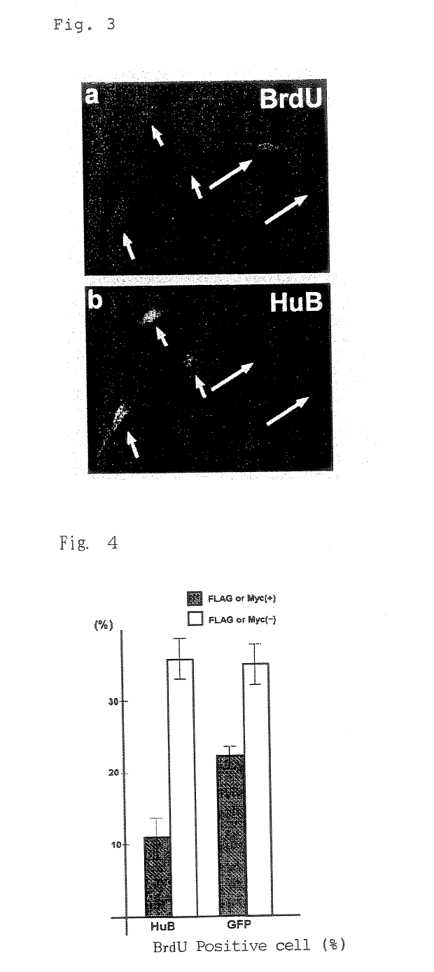

A plasmid described by Akamatsu et al. (PNAS 1999) was inserted in a vector pCXN2 and then transferred to SH-SY5Y cells through use of Lipofectamine Plus (BRL), to thereby forcedly induce expression of a modified gene obtained by adding a FLAG-tag to a mouse-derived, HuB-protein-encoding gene on its N-terminal side. The resultant cells were cultured in a 12-well dish containing cover glasses coated with Poly-L-lysine. Forty-eight hours after the transfer, the resultant HuB-incorporated cells were immunostained with an antibody for the FLAG-tag. Among the HuB-incorporated cells, a large number of cells with pyknosis were identified through Hoechst staining (see FIGS. 1a through 1c). In some of the HuB-incorporated cells with extended neurites—thus assuming the shape of a neuron—Hoechst staining also revealed the existence of cells with pyknosis (see FIGS. 1d through 1f).

The TUNEL positive rate of these incorporated cells was determined through the TUNEL method. The TUNEL positive rate of the HuB-incorporated cells was found to be about four times that of the control cells (GFP-Myc), indicating that apoptosis had been promoted (FIG. 2). G418 was added to the cells, and the incorporated cells were selected and observed for seven days. Observation revealed no cells that had further extended neurites and had differentiated, and no cells that had multiplied and had formed colonies.

### Example 2

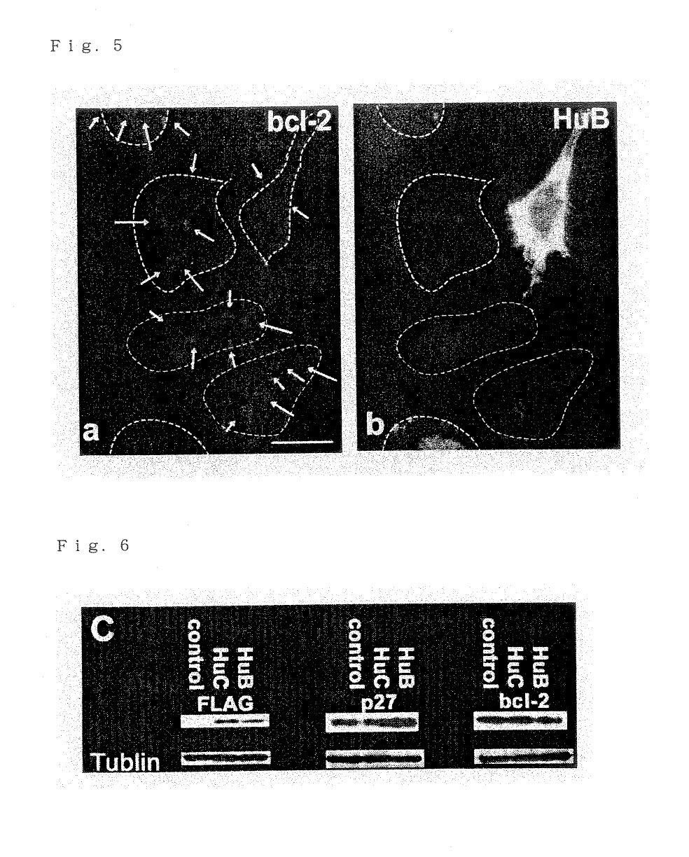

SH-SY5Y cells to which HuB had been incorporated were prepared. Starting from 36 hours after the gene transfer, stage-S cells underwent a labeling process with bromodeoxyuridine (BrdU) that had been added to the medium for 12 hours. Forty-eight hours after the gene transfer, the labeled cells were immunostained by use of anti-BrdU antibody and anti-FLAG (or anti-Myc) antibody (FIGS. 3a and 3b). The BrdU positive rate of the incorporated cells was calculated (FIG. 4). As a result, the HuB-incorporated SH-SY5Y cells were found to have incorporated about 50% less BrdU than the control cells. That is, overexpression of HuB was found to have halted multiplication of SH-SY5Y cells.

### Example 3

Bcl-2 is a differentiation marker which has been known to rise in level as differentiation proceeds. Twenty-four hours after the transfer of HuB, the resultant HuB-incorporated SH-SY5Y cells were immunostained with an antibody for Bcl-2. The results are shown in FIGS. 5a and 5b. The HuB-incorporated cells (FLAG-positive cells) were found to exhibit reduced Bcl-2 expression (represented by white arrows). The broken line represents the periphery of an individual cell. Separately, SH-SY5Y cells in which HuB, HuC, or a control (GFP-Myc) had been incorporated were subjected to immunoblotting through use of an antibody for p27 which has been reported to be bound to Hu protein, or Bcl-2 (FIG. 6). Through quantification performed on NIH-images, the HuC-incorporated cells were found to contain almost the same amount of p27 and Bcl-2, whereas the HuB-incorporated cells were found to contain about 40% more p27 and 35% less Bcl-2 than the control cells. In order to adjust quantification, an antibody for tubulin was employed.

These results indicate that enhanced p27 expression is related to the cell multiplication inhibitory effect of HuB, and that suppressed Bcl-2 expression is related to the cell death induction effect of HuB.

### Example 4

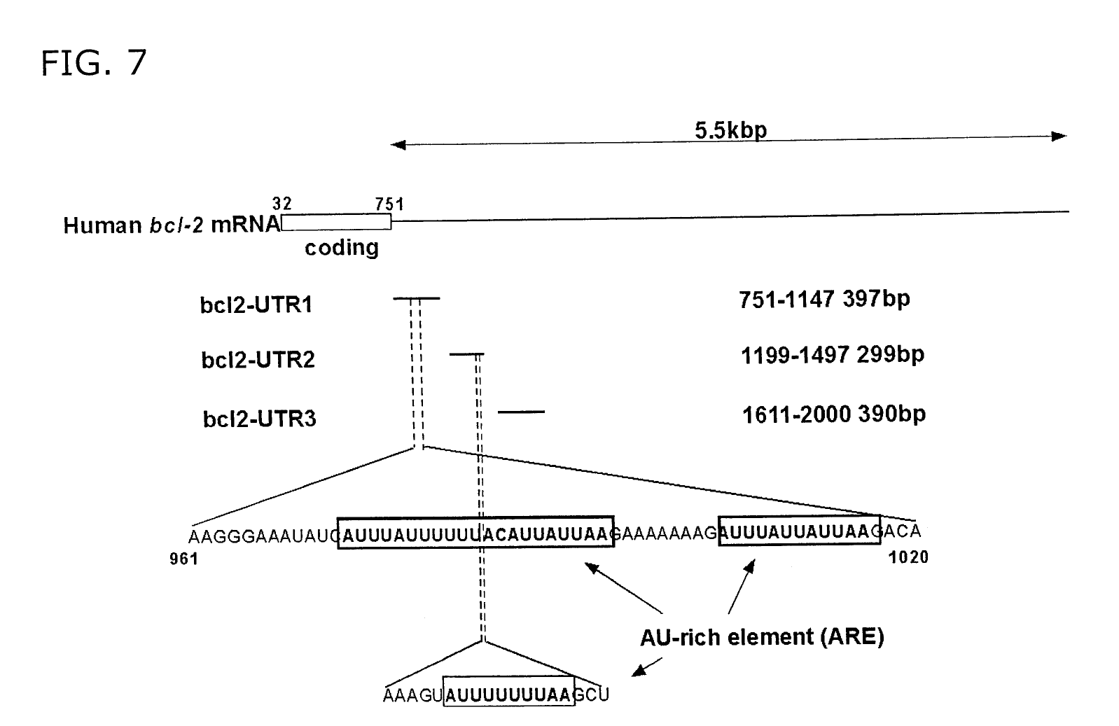

Human Bcl-2 gene has a long UTR portion (total length: about 5.5 kb) containing AU rich elements (ARE). The presence of an ARE between 961 bp and 1020 bp has been reported to deteriorate Bcl-2 mRNA stability (Schiavone et al., FASEB J, 2000 January; 14 (1): 174-84). Portions (UTR-1 and UTR-2) containing an ARE and a portion (UTR-3) containing no ARE (300 to 400 bp each, shown in FIG. 7) were subjected to subcloning.

### Example 5

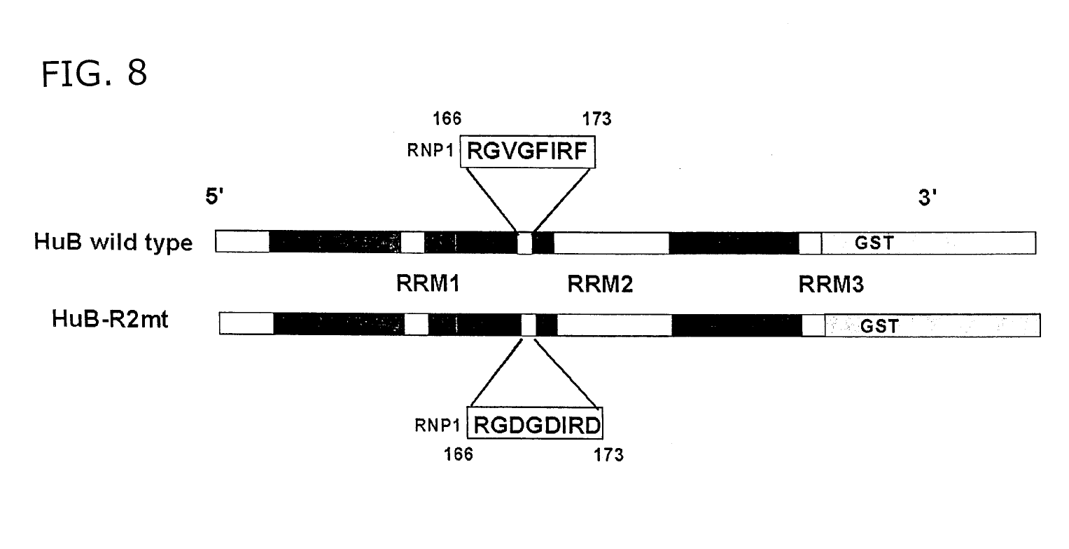

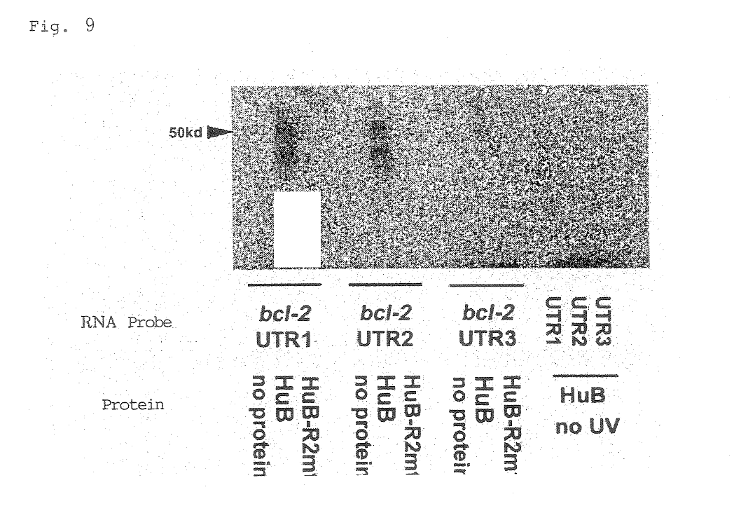

A mutant of HuB wild strain in which valine, phenylalanine, and phenylalanine in RNP1 of RRM2 are substituted by aspartics was prepared (FIG. 8). The mutant have been confirmed to have no binding ability to the target mRNA of HuB which belongs to the same Hu family, and thus to exhibit no differentiation induction effect.

### Example 6

pGEX-HuB or HuB-R2mt was expressed in E. coli BL21, followed by purification through use of glutathione Sepharose. The purified protein (200 ng) was mixed with each of Bcl-2 mRNA 3′UTR-1, -2, and -3 which had been labeled with ³²P-UTP, and the resultant mixture was subjected to UV-crosslinking for one minute through use of a Stratlinker. The product was electrophoresed by use of a 12.5% SDS-PAGE gel and detected through use of a BAS-5000 (FIG. 9).

HuB was found to be bound to UTR-1 and UTR-2, but not to UTR-3, which contains no AU-rich element. HuB-R2 was found not to be bound to any of UTR-1, UTR-2, and UTR-3.

These results reveal that HuB binds to mRNA of Bcl-2 containing AU-rich elements, and that this binding is lost when point mutation is introduced into RRM2.

The above Examples indicate that overexpression of Hu protein in SH-SY cells induces apoptosis of the cells. Although the PC12 cell, a cell strain derived from the same neural crest as the SH-SY cell, exhibited a phenotype of extension of neurites and halting of cell multiplication, and induced differentiation, the SH-SY cell did not exhibit differentiation. Contrarily, the SH-SY cell exhibited reduced expression of Bcl-2, a differentiation marker. Thus, Hu protein is considered to bind to the AU-rich element in the 3′UTR of Bcl-2 mRNA, whereby mRNA stability is deteriorated, leading to reduction in expression of Bcl-2. The reduction in expression of Bcl-2, which has apoptosis inhibitory effect, is considered to elevate the sensitivity of the SH-SY cells for apoptosis-inducing stimulus, resulting in promoting induction of apoptosis.

### INDUSTRIAL APPLICABILITY

The present invention provides a novel therapeutic method for neuroblastoma, which has been difficult to cure.

1-6. (canceled)

7. A method of treating a neuroblastoma in a subject, comprising
administering an effective amount of a nucleic acid encoding a mammalian HuB protein to the subject to treat the neuroblastoma, a nucleic acid which encodes a polypeptide having at least 80% homology to the HuB protein, or a nucleic acid which encodes a polypeptide having at least 90% homology to the HuB protein,
wherein the HuB protein comprises SEQ ID NO:3 and which has RNA binding activity.

8. A method of treating a neuroblastoma in a human subject, comprising
administering an effective amount of a nucleic acid encoding a human HuB protein, a nucleic acid which encodes a polypeptide having at least 80% homology to the HuB protein, or a nucleic acid which encodes a polypeptide having at least 90% homology to the HuB protein, to the subject to treat the neuroblastoma.

9. The method of claim 7, which comprises administering a nucleic acid encoding a human HuB protein.

10. The method of claim 8, which comprises administering a nucleic acid encoding a human HuB protein.

11. The method of claim 7, which comprises administering a nucleic acid encoding a polypeptide having at least 88% homology to the HuB protein.

12. The method of claim 7, which comprises administering a nucleic acid encoding a polypeptide having at least 80% homology to the HuB protein.

13. The method of claim 7, which comprises administering a nucleic acid encoding a polypeptide having at least 90% homology to the HuB protein.

14. The method of claim 8, which comprises administering a nucleic acid encoding a polypeptide having at least 90% homology to the HuB protein.
